(19) United States
(12) Patent Application Publication
(10) Pub. No.: US 2011/0186509 A1
(43) Pub. Date: Aug. 4, 2011

(54) PROCESSING OF WATER USING MICROORGANISMS

(75) Inventors: Jurgen Johann, Nussloch (DE); Wolfgang Neubrand, Unterwachingen (DE); Karl-Heinrich Engesser, Stuttgart (DE); Daniel Dobslaw, Stuttgart (DE)

(73) Assignee: CHRIST WATER TECHNOLOGY AG, Mondsee (AT)

(21) Appl. No.: 12/866,560

(22) PCT Filed: Feb. 6, 2009
(86) PCT No.: PCT/EP09/00824

§ 371 Date: Apr. 7, 2011

(30) Foreign Application Priority Data
Feb. 6, 2008 (DE) 10 2008 009 219.3

(51) Int. Cl. C02F 3/34; C12N 1/20; C12N 11/14; C02F 3/00; B65D 85/00

(52) U.S. Cl. 210/616; 435/252.1; 435/176; 210/601; 206/524.1; 210/151

(57) ABSTRACT

A method for microbiologically processing water includes treating the water in a bioreactor with microorganisms, wherein at least 70% of the microorganisms can break down 2-propanol and/or acetone.

### RELATED APPLICATIONS

This is a §371 of International Application No. PCT/EP2009/000824, with an international filing date of Feb. 6, 2009 (WO 2009/098066 A2, published Aug. 13, 2009), which is based on German Patent Application No. 10 2008 009 219.3, filed Feb. 6, 2008, the subject matter of which is incorporated.

### TECHNICAL FIELD

This disclosure relates to a method of microbiological processing of water, in particular waste waters from semiconductor production, and microorganisms which are suitable for use in such a method. In addition, the disclosure relates to a bioreactor, and also to a water processing plant having such microorganisms.

### BACKGROUND

Semiconductor production sites globally are among the largest industrial water consumers. The highest requirements are made of the quality of the water required in the semiconductor industry. For instance, the water must be essentially completely free of particles and dissolved inorganic and organic components. In recent years, attempts have increasingly been made to reprocess waste waters from semiconductor production (hereinafter also called “reclaim water”) and to establish a circulation procedure to minimize consumption of fresh water. Such a procedure offers advantages in many aspects:

Waste water from semiconductor production generally has a better defined composition than fresh water and, therefore, they may be treated more specifically.

Waste water from semiconductor production does not contain any significant amounts of inorganic components and hardness-forming agents (calcium or magnesium) and/or carbonates.

However, a problem for reprocessing, is the rather high fractions of organic components with which waste waters from the semiconductor industry are generally admixed. Such waste waters customarily have a total organic carbon (TOC) content in the range between 500 ppb and 3000 ppb. The organic components are predominantly residues of synthetic organic solvents. Compared with natural organic water components such as humic acids or biopolymers, these have a lower molar mass. In addition, they are polar and virtually indisociable. As an example thereof 2-propanol may be mentioned, which frequently makes up the largest fraction of the impurities.

Therefore, if the production of ultrapure water for the semiconductor industry starts from reclaim water (and/or from a mixture of fresh water and reclaim water), then special measures must be taken to remove the organic components from the water which is to be processed.

Some procedures are known which are devoted to this special problem. For instance, biological method stages started to be integrated a relatively long time ago in the process of ultrapure water processing (Golsham M. and Schmitt S.: Water reuse and reclaim operations at Hyundai semiconductor America, Ultrapure Water 05, 2001, 34-38; JP 61111198; JP 2002-2336886). According to the procedures described there, microorganisms are either directly suspended in the process water or applied to solid support materials which are subsequently brought into contact with the process water. The microorganisms take up organic compounds and also oxygen, nutrients and dissolved minerals from the waste water and convert these impurities to inert or easily removable compounds. Organic carbon is preferably mineralized in that case. Sewage sludge is generally used as a source of the microorganisms, with which sewage sludge suitable reactors in the ultrapure water processing process are inoculated.

In fact, a treatment with microorganisms can lead to a sometimes very significant reduction of the fraction of organic components. However, it has been observed that efficiency and reliability of such measures are frequently subject to large fluctuations. The results obtained have not always been reproducible. Furthermore, in many cases there is also the risk that the water treatment plants are microbially inoculated in an uncontrolled manner, in particular also with pathogenic microorganisms.

It could therefore be helpful to provide a technical solution for the abovementioned problem. The solution should efficiently and reproducibly enable, in particular, purification of waste water from the semiconductor industry having a high fraction of low-molecular-weight organic impurities such as 2-propanol and acetone. In this case they should be configured simply and inexpensively.

### SUMMARY

We provide a method for microbiologically processing water including treating the water in a bioreactor with microorganisms, wherein at least 70% of the microorganisms can break down 2-propanol and/or acetone.

We also provide a bioreactor for water processing including microorganisms of which at least 70% can break down 2-propanol and/or acetone.

We also provide a microorganism for use in a method, a bioreactor, or in a water processing plant which is a bacterial strain of the bacterial genus Xanthobacter DSM No. 19987 or a mutant thereof.

We further provide a microorganism for use in a method, a bioreactor, or in a water processing plant which is a bacterial strain of the bacterial genus Rhodococcus DSM No. 19985 or a mutant thereof.

We also provide a microorganism for use in a method, a bioreactor, or in a water processing plant which is a bacterial strain of the bacterial genus Paracoccus DSM No. 19986 or a mutant thereof.

### BRIEF DESCRIPTION OF THE DRAWING

Details on the features described and also other features result from the following description in combination with the drawing. In this case the individual features can be effected each alone or in combination with one another.

FIG. 1 shows a flow chart of a subsection of a water processing plant for treating reclaim water.

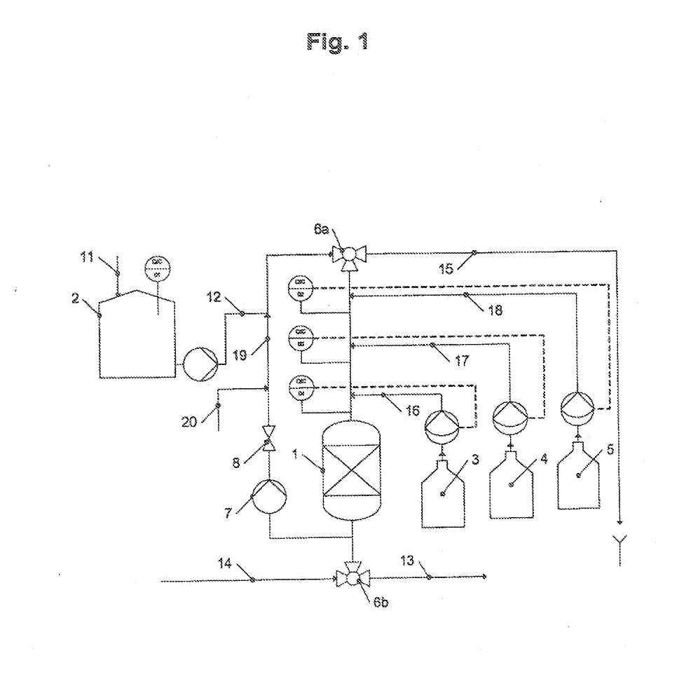

### DETAILED DESCRIPTION

Our method serves for the microbiological processing of water, in particular waste waters from semiconductor production. It comprises the water which is to be processed being treated in a bioreactor with microorganisms. Particularly preferably, the water which is to be processed is treated with microorganisms of which at least 70% can break down 2-propanol and/or acetone. The microorganisms are therefore specialized in the breakdown of those compounds and certainly not completely undefined.

The microorganisms can in principle here be not only a single bacterial strain, but also bacterial cultures which comprise a plurality of bacterial strains or even strains from different bacterial genera. The critical factor is that the fraction of microorganisms which break down 2-propanol and/or acetone does not fall below the selected limit. Preferably, the fraction of microorganisms breaking down 2-propanol and/or acetone is greater than 80%, in particular greater than 90%, particularly preferably virtually 100%.

As mentioned at the outset, it is known to use microorganisms from sewage sludge for breaking down low-molecular-weight organic compounds. However, this is in no way a targeted selection of microorganisms. The fraction of microorganisms which break down specifically determined impurities, is in all procedures known to date rather determined by chance.

In contrast, as per our process, preferably microorganisms are used in a targeted manner, which microorganisms have growth and breakdown kinetics matched to specific impurities, namely to 2-propanol and/or acetone. By this measure, organic carbon contained in reclaim water was reproducibly and extremely efficiently removed. Generally—starting from reclaim water having a TOC value between 500 ppb and 3000, ppb—breakdown rates of 80% or more were always observed. Such a reliable solution for removing organic carbon contained in reclaim water is to date not known.

In our preparatory work, diverse bacterial stains of different genera were investigated for their suitability. Particularly suitable microorganisms were identified within the bacterial genus Xanthobacter. Correspondingly, in the context of our method, the water which is to be processed is treated particularly preferably with at least one bacterial strain of the bacterial genus Xanthobacter and/or a mutant thereof.

The strain which was deposited under the number DSM No. 19987 at the deutsche Sammlung von Mikroorganismen und Zellkulturen GmbH (DSMZ [German Collection of Microorganisms and Cell Cultures]) has proved to be particularly suitable.

On standard media, this strain generally formed transparent whiteish matt colonies having a smooth rim, round growth, homogeneous structure and flat profile. Diplococci having coccoid cell shape and rounded corners, a width of approximately 0.8 μm and a length of approximately 1.2 μm were observed.

Precise phylogenetic assignment was made by determining the nucleic acid sequence by means of direct sequencing of the PCR-amplified 16S rRNA. Thereafter, the sequence data were compared with the known sequences of representatives of the genus Xanthobacter. The highest sequence agreement of 99.5% was found with Xanthobacter flavus (DSM 338).

In addition, the water which is to be processed can also be treated with at least one bacterial strain of the bacterial genus Rhodococcus and/or a mutant thereof. Such bacterial strains have also proved to be very suitable for use in our method.

In particular, the strain which was deposited at the deutsche Sammlung von Mikroorganismen und Zellkulturen GmbH (DSMZ) under the DSM No. 19985 was identified as particularly suitable.

For this strain, the nucleic acid sequence was determined by direct sequencing of the PCR-amplified 16S rRNA. By comparing the sequence data with the sequences of known representatives of the genus Rhodococcus, the strain was identified as a representative of suborder Rhodococcus ruber.

Bacterial strains of the bacterial genus Paracoccus and/or mutants thereof are likewise highly suitable for use in our method. In particular, very good results were achieved using the bacterial strain which was deposited under the DSM No. 19986 at the deutsche Sammlung von Mikroorganismen and Zellkulturen GmbH (DSMZ).

The above bacterial strains identified as preferred can be used not only individually, but also in combination in our method. They can also be present in mixed cultures with one or more other bacterial strains which, if appropriate, are not able to break down 2-propanol and/or acetone.

Preferably, the fraction of microorganisms which break down 2-propanol and/or acetone is then always above the preferred limit values. Preferably, the microorganisms used are exclusively one or more of the strains identified as preferred.

The abovementioned bioreactor is in the simplest case a container in which the water which is to be treated is brought into contact with the microorganisms, for example, by suspending the microorganisms in the water which is to be processed.

However, particularly preferably the bioreactor used is an aerated or non-aerated filtration unit which preferably has a particulate filter material. Equipped in such a manner, the bioreactor can fulfil simultaneously a plurality of functions. First, low-molecular-weight organic components such as 2-propanol and/or acetone can be broken down in it. Second, it can act as a filter to remove particles from the water which is to be processed.

Particulate filter materials, owing to their large surface area, offer good conditions for colonization by microorganisms. Particularly preferably, the microorganisms are immobilized on the filter material in a bioreactor constructed as a filtration unit. The microorganisms can form a continuous film on the particles and/or in pores of the filter material.

In the literature, generally, a flow path from bottom to top is reported as a preferred mode of operation of biofilters (see, e.g., EP 585036, JP 7284799, JP 6063592 and JP 62065792). When the flow passes through a biofilter from bottom to top, however, microorganisms can easily be washed out with the process water. Consequently, process stages downstream can be microbially inoculated. Known bioreactors, for this reason, are frequently combined with additional filtration stages which are connected downstream of the biofilter (see, e.g., JP 63185494, JP 8197094 and JP 9155371). However, such solutions are complex in terms of equipment and are expensive.

It is preferred that the water which is to be processed is passed as a waste stream through such a bioreactor which is constructed as a filtration unit. In this manner, it was ensured that elevated concentrations of microorganisms did not exit from the filtration unit. Reclaim water, for example, downstream of the biological treatment can be introduced directly into the filtrate water tank of a water'processing plant.

A bioreactor serves specifically for water processing, in particular, for processing waste water from the semiconductor industry and is suitable, in particular, for use in our method. It has microorganisms which can break down 2-propanol and/or acetone.

Preferably, it has microorganisms of which at least 70%, preferably more than 80%, in particular more than 90%, particularly preferably virtually 100%, can break down 2-propanol and/or acetone.

As mentioned above, bacterial strains of the bacterial genus Xanthobacter and/or Rhodococcus and/or Paracoccus or mutants thereof are particularly suitable as microorganisms. The above description of microorganisms which can preferably be used is hereby explicitly incorporated herein by reference.

Accordingly, the bioreactor is preferably constructed as an aerated or non-aerated filtration unit, wherein the filtration unit has a particulate filter material on which the microorganisms are immobilized. The advantageous bifunctionality of such a bioreactor has already been considered above.

The particulate filter material is particularly preferably activated carbon. However, other materials, for example structured or irregular packings of plastics such as polystyrene, sand, expanded clay or anthracite, can also be very highly suitable. Preferably, the particulate filter material can be porous, in particular have pores having a size which exceeds the size of the microorganisms at least by a factor of 10.

Activated carbon has a significant adsorption capacity for organic solvents, in particular for 2-propanol and acetone. By loading the filter material with these solvents, a reservoir of substrate is formed to which the microbiological culture is specifically adapted. In operation, fluctuations in rate and composition of the waste water to be treated can be compensated for.

We further provide a water processing plant which is characterized in that it has a bioreactor. Water processing plants of the type in question for producing ultrapure water, in particular for purposes of the semiconductor industry, are sufficiently known and, therefore, do not need to be described in detail.

In agreement with the above details, a microorganism for use in our method is distinguished in particular in that it is a bacterial strain of the bacterial genus Xanthobacter having the DSM No. 19987 or a mutant thereof. A further microorganism is distinguished in that it is a bacterial strain of the bacterial genus Rhodococcus, having the DSM No. 19985 or a mutant thereof. A third microorganism belongs to the genus Paracoccus and was deposited at the DSMZ under the DSM No. 19986.

Preferably, the microorganisms can be immobilized on a support material such as activated carbon. Further suitable supports have been mentioned above as filter materials.

In addition, it can be preferred that the microorganisms are present packed in a water-tight, air-permeable container. This is because, surprisingly, it is proved that microorganisms immobilized in particular on activated carbon can be preserved even over relatively long time periods, provided they are packed in containers which ensure sufficient oxygen supply to the microorganisms. Such containers are, for example, flat bags made of plastic film, preferably polyethylene, in particular having a film thickness of approximately 0.05 mm. These have sufficient air permeability to ensure the oxygen supply of enclosed microorganisms over relatively long time periods of up to several weeks. The nutrient requirement of the microorganisms in this time period can be covered, for example, by 2-propanol stored by adsorption on the support material.

The bags can be closed in a sterile and water-tight manner. In this manner the immobilized microorganisms can be stored over relatively long time periods and transported over large distances to the site of use. At the site of use, e.g., the fixed bed of a filtration unit, for example a fixed bed of activated carbon, can very simply be inoculated with the immobilized microorganisms (conversion of a simple filtration unit into a bioreactor). Addition of one part by volume of activated carbon having microorganisms immobilized thereon to generally 50 to 10 000 parts by volume of fixed bed is generally sufficient.

An important condition for the rapid multiplication of microorganisms in the bioreactor is the addition of nutrient salts to the reactor feed. The nutrient salts are preferably added until the bioreactor has reached its full output. The nutrient salts preferably contain the required macro elements (in particular N, P, S, Na, Ca and Mg) and also trace elements (in particular Fe, Cu, Cr, Co and Zn). The addition of nutrient salts is adjusted, in particular, in such a manner that in the reactor feed the mole-based concentrations of the elements C, N and P are in the ratio C:N:P=100:10:1 to one another.

Several advantages result from the availability of the microorganisms according to the invention:

The availability at short notice of microorganisms, e.g. for inoculating activated carbon fixed beds, is ensured at all times.

The microorganisms can be provided in a constant and controlled quality.

The above-described microorganisms are not pathogenic, and their use is therefore risk-free.

The requirements of the biomatter regulation are complied with in full.

The bioreactors can be activated by personnel who are not specially trained.

Installation and operation of separate fermentation plants at the site of the bioreactor are avoided.

The bioreactor can be very rapidly put into operation, its start up-time is greatly shortened.

The FIGURE shows the water tank 2 having the feed 11 for reclaim water. The water tank 2 has a means for controlling the pH of the reclaim water which is contained. Preferably, the pH in the water tank is set to values between 4 and 9, in particular between 7 and 8. From the water tank 2, a line 12 leads to the bioreactor 1, in which there is situated a fixed bed of activated carbon, the surface of which is colonized by microorganisms of the strain having the DSM No. 19985. Before the reclaim water is introduced into the bioreactor 1, the reclaim water can be admixed, if needed, with electrolyte from the reservoir tank 3 via the line 16. This is necessary, if appropriate, since the reclaim water must not be completely salt free to ensure in the long term the survival of the microorganisms in the bioreactor. In the reservoir tanks 4 and 5 there are situated 2-propanol and nutrient salts. From these, substrate (e.g. 2-propanol) and nutrients can be added to the reclaim water via lines 17 and 18, which can, if appropriate, be necessary in the activation phase or for compensating for Concentration variations in operation. If required, the water which is to be processed can be circulated and also aerated. The latter can be necessary during start-up of the biological activation. The valves 6a and 6b serve for switching over between production operation, circulation and backwash. The latter can be necessary, e.g., when the fixed bed of the bioreactor is blocked by dirt particles from the water which is to be processed. Furthermore, the water processing plant has a circulation pump 7 for circulation operation and also a shut-off valve 8 for the circulated stream. The outlet for water which is treated in the bioreactor in the production mode and also the feed of the backwash stream are indicated by the reference signs 13 and 14.

1. A method for microbiologically processing water, comprising treating the water in a bioreactor with microorganisms, wherein at least 70% of the microorganisms can break down 2-propanol and/or acetone.

2. The method according to claim 1, wherein the water is treated with a bacterial strain of the bacterial genus Xanthobacter and/or a mutant thereof.

3. The method according to claim 2, wherein the bacterial strain is Xanthobacter flavus DSM No. 19987.

4. The method according to claim 1, wherein the water is treated with a bacterial strain of the bacterial genus Rhodococcus and/or a mutant thereof.

5. The method according to claim 4, wherein the bacterial strain is Rhodococcus ruber DMS No. 19985.

6. The method according to claim 1, wherein the water is treated with a bacterial strain of the bacterial genus Paracoccus and/or a mutant thereof.

7. The method according to claim 6, wherein the bacterial strain is strain DMS No. 19986.

8. The method according to claim 1, wherein the bioreactor is a filtration unit having a particulate filter material.

9. The method according to claim 8, wherein the microorganisms are immobilized on the filter material.

10. The method according to claim 8, wherein the water is waste stream passed through the filtration unit.

11. A bioreactor for water processing comprising microorganisms of which at least 70% can break down 2-propanol and/or acetone.

12. The bioreactor according to claim 11, wherein the microorganism comprises at least one bacterial strain of the bacterial genera Xanthobacter and/or Rhodococcus and/or Paracoccus and/or at least one mutant thereof.

13. The bioreactor according to claim 11, constructed as a filtration unit, wherein the filtration unit has a particulate filter material on which the microorganisms are immobilized.

14. The bioreactor according to claim 13, wherein the particulate filter material is activated carbon.

15. A water processing plant comprising the bioreactor according to claim 11.

16. A microorganism for use in a method, a bioreactor, or in a water processing plant which is a bacterial strain of the bacterial genus Xanthobacter DSM No. 19987 or a mutant thereof.

17. A microorganism for use in a method, a bioreactor, or in a water processing plant which is a bacterial strain of the bacterial genus Rhodococcus DSM No. 19985 or a mutant thereof.

18. A microorganism for use in a method, a bioreactor, or in a water processing plant which is a bacterial strain of the bacterial genus Paracoccus DSM No. 19986 or a mutant thereof.

19. The microorganism according to claim 16, immobilized on an activated carbon support.

20. The microorganism according to claim 16, packed in a water-tight, air-permeable container.

21. (canceled)
